(19) United States
(12) Patent Application Publication
(10) Pub. No.: US 2025/0114353 A1
(43) Pub. Date: Apr. 10, 2025

(54) Combination Treatment Regimens - SMARCA2 Degrader With Gemcitabine

(71) Applicant: PRELUDE THERAPEUTICS INCORPORATED, Wilmington, DE (US)

(72) Inventors: Koichi Ito, Philadelphia, PA (US); Michael Hulse, Doylestown, PA (US); Peggy Scherle, Wayne, PA (US); Krishna Vaddi, Chadds Ford, PA (US)

(21) Appl. No.: 18/906,330

(22) Filed: Oct. 4, 2024

Related U.S. Application Data
(60) Provisional application No. 63/587,767, filed on Oct. 4, 2023.
(60) Provisional application No. 63/594,108, filed on Oct. 30, 2023.

(51) Int. Cl. A61K 31/5025; A61K 31/282; A61K 31/337; A61K 31/7068; A61K 33/243; A61P 35/00

(52) U.S. Cl. CPC A61K 31/5025; A61K 31/282; A61K 31/337; A61K 31/7068; A61K 33/243; A61P 35/00

(57) ABSTRACT

Disclosed are methods of treating cancer comprising administering to a subject a treatment regimen comprising a compound of Formula (I):

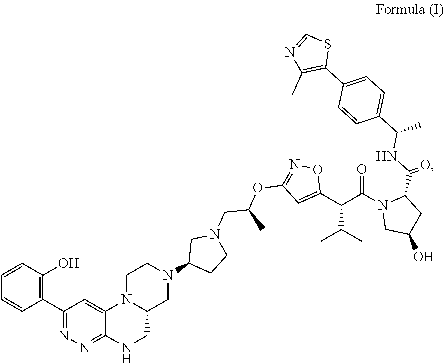 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

or a pharmaceutically acceptable salt thereof, and gemcitabine, or a pharmaceutically acceptable salt thereof.

### CROSS-REFERENCE TO RELATED APPLICATIONS

This application claims the benefit of U.S. Provisional Application No. 63/587,767, filed Oct. 4, 2023; and U.S. Provisional Application No. 63/594,108, filed Oct. 30, 2023. Each of the aforementioned applications is incorporated by reference herein in its entirety.

### TECHNICAL FIELD

The disclosure pertains to methods of treating cancer comprising administering to a subject a treatment regimen comprising a SMARCA2 degrader and gemcitabine, or a pharmaceutically acceptable salt thereof.

### BACKGROUND

SMARCA2 and SMARCA4 are the core catalytic subunits of the SWI/SNF complexes, which play an important role in controlling gene expression by remodeling chromatin. SMARCA4 is mutated in multiple cancers and SMARCA4-deficient cancer cells can become highly dependent on SMARCA2 for their survival. Therefore, targeting SMARCA2 in SMARCA4-deleted cancers using selective SMARCA2 degraders induces synthetic lethality, while sparing SMARCA4 wild-type normal cells. Cell lines with SMARCA4 damaging mutation or low expression show high SMARCA2 gene dependency scores, suggesting the synthetic lethal relationship of targeting SMARCA2 and SMARCA4-deficiency. SMARCA4 mutations occur in different types of cancer including lung adenocarcinoma, skin cancer/melanoma, uterine/endometrioid adenocarcinoma, bladder cancer, colon adenocarcinoma, among others. In addition to damaging mutation (“loss of SMARCA4 protein”), some tumors express SMARCA4 hotspot missense mutations near its ATP binding site or DNA binding site that likely alters biological function of SMARAC4.

The compound of Formula (I) is a SMARCA2 degrader:

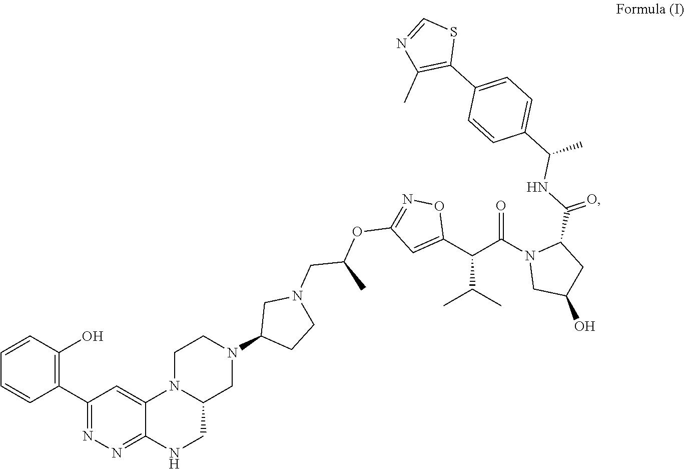 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

that was disclosed in WO2021252666. Formula (I) regulates cancer cell proliferation and growth through cell cycle arrest and DNA replication inhibition in SMARCA4 mutated cancer cells.

Gemcitabine is a nucleoside analogue of the antimetabolite class of anti cancer agents. Gemcitabine has the chemical name 4-amino-1-((2R,4R,5R)-3,3-difluoro-4-hydroxy-5-(hydroxymethyl)tetrahydrofuran-2-yl)pyrimidin-2(1H)-one, and the chemical structure:

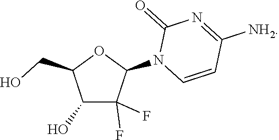 [SMILES: Nc1ccn([C@@H]2O[C@H](CO)[C@@H](O)C2(F)F)c(=O)n1]

The monohydrochloride salt of gemcitabine is marketed under the tradename GEMZAR®.

### SUMMARY

The disclosure provides methods of treating cancer in a subject in need thereof, comprising administering to the subject a treatment regimen comprising:
(a) a compound of Formula (I):

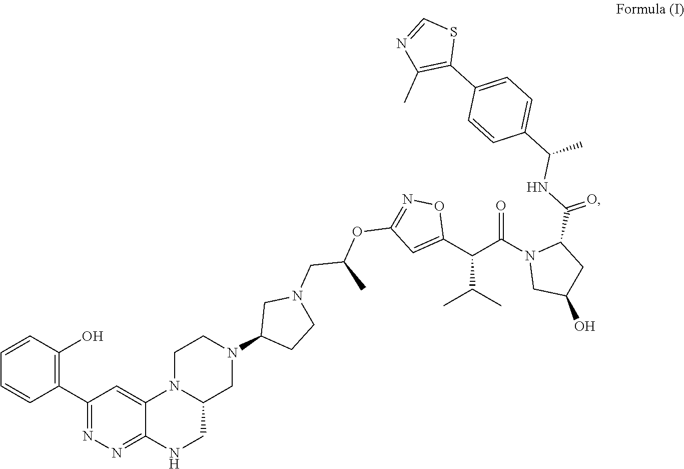 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

or a pharmaceutically acceptable salt thereof; and
(b) gemcitabine, or a pharmaceutically acceptable salt thereof.

In some aspects, the cancer is a SMARCA4-deleted cancer.

### BRIEF DESCRIPTION OF THE DRAWINGS

FIG. 1 shows that the Formula (I) combination with gemcitabine resulted in deeper tumor growth inhibition (TGI) compared to each single agent alone in the SMARCA4-deficient NCI-H838 Lung Cancer Xenograft Model in Mice. NOD/SCID female mice were inoculated subcutaneously with NCI-H838-#5 cells (5×10⁶) into the right flank in PBS 0.1 ml mixed with Matrigel. When tumor volumes reached approximately 100-150 mm³, mice were randomized and dosed with Formula (I) at 100 mg/kg s.c. Q3D; gemcitabine 100 mg/kg i.v. BIW; or Formula (I) at 100 mg/kg s.c. Q3D+gemcitabine 100 mg/kg i.v. BIW. *P<0.05, **P<0.01, versus vehicle (two-tailed unpaired Mann-Whitney test).

### DETAILED DESCRIPTION OF ILLUSTRATIVE EMBODIMENTS

The disclosure may be more fully appreciated by reference to the following description, including the following definitions and examples. Certain features of the disclosed compositions and methods which are described herein in the context of separate aspects, may also be provided in combination in a single aspect. Alternatively, various features of the disclosed compositions and methods that are, for brevity, described in the context of a single aspect, may also be provided separately or in any sub combination.

Unless otherwise defined, all technical and scientific terms used herein have the same meaning as commonly understood by one of ordinary skill in the art to which this disclosure pertains. The terminology used in the description is for describing particular embodiments only and is not intended to be limiting of the disclosure.

The articles “a,” “an,” and “the” as used herein and in the appended claims are used herein to refer to one or to more than one (e.g., to at least one) of the grammatical object of the article unless the context clearly indicates otherwise. By way of example, “an element” means one element or more than one element.

Where a range of values is provided, it is to be understood that each intervening value, to the tenth of the unit of the lower limit unless the context clearly dictates otherwise, between the upper and lower limit of that range and any other stated or intervening value in that stated range is encompassed within the disclosure. The upper and lower limits of these smaller ranges may independently be included in the smaller ranges is also encompassed within the disclosure, subject to any specifically excluded limit in the stated range. Where the stated range includes one or both of the limits, ranges excluding either or both of those included limits are also included in the disclosure.

When a range of values is expressed, it is to be understood that the explicitly stated upper and lower limits of the range and all intervening individual values, and all intervening subranges are encompassed by the range. All ranges are inclusive and combinable.

When values are expressed herein as approximations, such as by use of the antecedent “about,” it is understood that the particular value forms another embodiment. In some embodiments, “about” refers to a value with ±10% of the particular value.

The term “administering,” when used in the context of administering a therapeutic agent to a subject, refers to introducing the therapeutic agent into the subject's body. For example, therapeutic agents may be introduced into a subject's body orally, nasally, subcutaneously, intravenously, intravesically, intramuscularly, transdermally, vaginally, rectally or in any combination thereof.

“Treating” or “treatment” of any disease or disorder refers, in one embodiment, to ameliorating the disease or disorder (e.g., arresting or reducing the development of the disease or at least one of the clinical symptoms thereof). In another embodiment “treating” or “treatment” refers to ameliorating at least one physical parameter, which may not be discernible by the subject. In yet another embodiment, “treating” or “treatment” refers to modulating the disease or disorder, either physically, (e.g., stabilization of a discernible symptom), physiologically, (e.g., stabilization of a physical parameter), or both. In yet another embodiment, “treating” or “treatment” refers to delaying the onset of the disease or disorder.

The term “subject” is used herein to describe an animal, for example, a mammal, to whom treatment with the treatment regimen according to the disclosure is provided. In some aspects, the mammal is a human to whom treatment is provided. In other aspects, the mammal is a non-human to whom treatment is provided.

“Pharmaceutically acceptable” means approved or approvable by a regulatory agency of the United States Federal government or a state government or the corresponding agency in countries other than the United States, or that is listed in the U.S. Pharmacopoeia or other generally recognized pharmacopoeia for use in animals, e.g., in humans.

“Pharmaceutically acceptable salt” refers to a salt of a compound of the disclosure that is pharmaceutically acceptable and that possesses the desired pharmacological activity of the parent compound. In particular, such salts are non-toxic may be inorganic or organic acid addition salts and base addition salts. Specifically, such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethane-sulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, N-methylglucamine and the like. Salts further include, by way of example only, sodium, potassium, calcium, magnesium, ammonium, tetraalkylammonium, and the like; and when the compound contains a basic functionality, salts of non-toxic organic or inorganic acids, such as hydrochloride, hydrobromide, tartrate, mesylate, acetate, maleate, oxalate, and the like.

A “pharmaceutically acceptable excipient” refers to a substance that is non-toxic, biologically tolerable, and otherwise biologically suitable for administration to a subject, such as an inert substance, added to a pharmacological composition or otherwise used as a vehicle, carrier, or diluent to facilitate administration of an agent and that is compatible therewith. Examples of excipients include calcium carbonate, calcium phosphate, various sugars and types of starch, cellulose derivatives, gelatin, vegetable oils, and polyethylene glycols. See for example, Remington, J. P. (2020). Remington, the science and practice of pharmacy, Elsevier Science.

As used herein, the term “treatment regimen” refers to coordinated dosages and administration timings of each of Formula (I) (or pharmaceutically acceptable salt thereof) and the taxane anticancer agent agent such that Formula (I) (or pharmaceutically acceptable salt thereof) and the taxane anticancer agent are administered to the subject during an overlapping period of time. Thus, the “treatment regimen” of the disclosure encompasses both concurrent administration of the compound of Formula (I) (or a pharmaceutically acceptable salt thereof) and the taxane anticancer agent; and sequential administration of the compound of Formula (I) (or a pharmaceutically acceptable salt thereof) and a taxane anticancer agent. Moreover, the treatment regimen encompasses administration of the compound of Formula (I) (or a pharmaceutically acceptable salt thereof) and a taxane anticancer agent on the same day; on different days; and any permutation thereof.

In some aspects, the disclosure is directed to methods of treating cancer in a subject in need thereof, comprising administering to the subject a treatment regimen comprising:
(a) a compound of Formula (I):

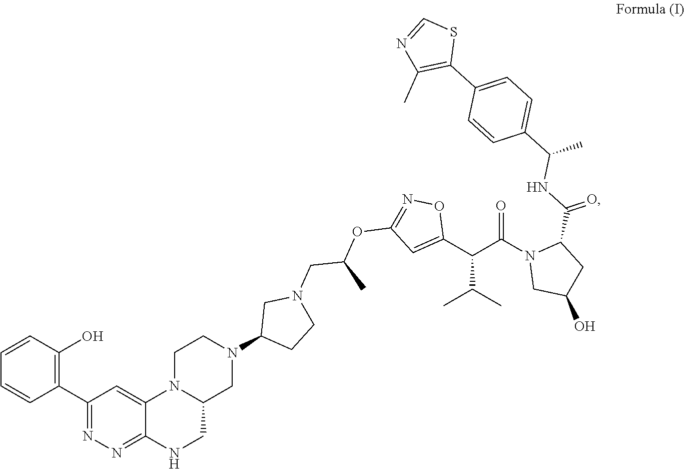 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]

or a pharmaceutically acceptable salt thereof; and
(b) gemcitabine, or a pharmaceutically acceptable salt thereof.

Methods of making the compound of Formula (I) are known in the art, and include those set forth in WO2021252666.

In some embodiments of the disclosed methods, the compound of Formula (I), or a pharmaceutically acceptable salt thereof, and the gemcitabine (or pharmaceutically acceptable salt thereof) are administered concurrently or sequentially.

In some embodiments of the disclosed methods, the compound of Formula (I), or a pharmaceutically acceptable salt thereof, and the gemcitabine (or pharmaceutically acceptable salt thereof) are administered concurrently.

In other embodiments of the disclosed methods, the compound of Formula (I), or a pharmaceutically acceptable salt thereof, and the gemcitabine (or pharmaceutically acceptable salt thereof) are administered sequentially.

In some aspects, the subject is administered the compound of Formula (I), or a pharmaceutically acceptable salt of a compound of Formula (I) (on a Formula (I) basis), in an amount of about 20 mg-about 1000 mg per dose, such as, for example, about 20 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 105 mg, about 110 mg, about 115 mg, about 120 mg, about 125 mg, about 130 mg, about 135 mg, about 140 mg, about 145 mg, about 150 mg, about 155 mg, about 160 mg, about 165 mg, about 170 mg, about 175 mg, about 180 mg, about 185 mg, about 190 mg, about 195 mg, about 200 mg, about 200 mg, about 205 mg, about 210 mg, about 215 mg, about 220 mg, about 225 mg, about 230 mg, about 235 mg, about 240 mg, about 245 mg, about 250 mg, about 255 mg, about 260 mg, about 265 mg, about 270 mg, about 275 mg, about 280 mg, about 285 mg, about 290 mg, about 295 mg, about 300 mg, about 305 mg, about 310 mg, about 315 mg, about 320 mg, about 325 mg, about 330 mg, about 335 mg, about 340 mg, about 345 mg, about 350 mg, about 355 mg, about 360 mg, about 365 mg, about 370 mg, about 375 mg, about 380 mg, about 385 mg, about 390 mg, about 395 mg, about 400 mg, about 405 mg, about 410 mg, about 415 mg, about 420 mg, about 425 mg, about 430 mg, about 435 mg, about 440 mg, about 445 mg, about 450 mg, about 455 mg, about 460 mg, about 465 mg, about 470 mg, about 475 mg, about 480 mg, about 485 mg, about 490 mg, about 495 mg, about 500 mg, about 505 mg, about 510 mg, about 515 mg, about 520 mg, about 525 mg, about 530 mg, about 535 mg, about 540 mg, about 545 mg, about 550 mg, about 555 mg, about 560 mg, about 565 mg, about 570 mg, about 575 mg, about 580 mg, about 585 mg, about 590 mg, about 595 mg, about 600 mg, about 605 mg, about 610 mg, about 615 mg, about 620 mg, about 625 mg, about 630 mg, about 635 mg, about 640 mg, about 645 mg, about 650 mg, about 655 mg, about 660 mg, about 665 mg, about 670 mg, about 675 mg, about 680 mg, about 685 mg, about 690 mg, about 695 mg, about 700 mg, about 705 mg, about 710 mg, about 715 mg, about 720 mg, about 725 mg, about 730 mg, about 735 mg, about 740 mg, about 745 mg, about 750 mg, about 755 mg, about 760 mg, about 765 mg, about 770 mg, about 775 mg, about 780 mg, about 785 mg, about 790 mg, about 795 mg, about 800 mg, about 805 mg, about 810 mg, about 815 mg, about 820 mg, about 825 mg, about 830 mg, about 835 mg, about 840 mg, about 845 mg, about 850 mg, about 855 mg, about 860 mg, about 865 mg, about 870 mg, about 875 mg, about 880 mg, about 885 mg, about 890 mg, about 895 mg, about 900 mg, about 905 mg, about 910 mg, about 915 mg, about 920 mg, about 925 mg, about 930 mg, about 935 mg, about 940 mg, about 945 mg, about 950 mg, about 955 mg, about 960 mg, about 965 mg, about 970 mg, about 975 mg, about 980 mg, about 985 mg, about 990 mg, about 995 mg, or about 1000 mg per dose.

In some aspects, the subject is administered the compound of Formula (I), or a pharmaceutically acceptable salt of a compound of Formula (I) (on a Formula (I) basis), in an amount of 20 mg-800 mg per dose, such as, for example, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 305 mg, 310 mg, 315 mg, 320 mg, 325 mg, 330 mg, 335 mg, 340 mg, 345 mg, 350 mg, 355 mg, 360 mg, 365 mg, 370 mg, 375 mg, 380 mg, 385 mg, 390 mg, 395 mg, 400 mg, 405 mg, 410 mg, 415 mg, 420 mg, 425 mg, 430 mg, 435 mg, 440 mg, 445 mg, 450 mg, 455 mg, 460 mg, 465 mg, 470 mg, 475 mg, 480 mg, 485 mg, 490 mg, 495 mg, 500 mg, 505 mg, 510 mg, 515 mg, 520 mg, 525 mg, 530 mg, 535 mg, 540 mg, 545 mg, 550 mg, 555 mg, 560 mg, 565 mg, 570 mg, 575 mg, 580 mg, 585 mg, 590 mg, 595 mg, 600 mg, 605 mg, 610 mg, 615 mg, 620 mg, 625 mg, 630 mg, 635 mg, 640 mg, 645 mg, 650 mg, 655 mg, 660 mg, 665 mg, 670 mg, 675 mg, 680 mg, 685 mg, 690 mg, 695 mg, 700 mg, 705 mg, 710 mg, 715 mg, 720 mg, 725 mg, 730 mg, 735 mg, 740 mg, 745 mg, 750 mg, 755 mg, 760 mg, 765 mg, 770 mg, 775 mg, 780 mg, 785 mg, 790 mg, 795 mg, or 800 mg per dose.

In some aspects, the subject is administered the compound of Formula (I), or a pharmaceutically acceptable salt of a compound of Formula (I) (on a Formula (I) basis), in an amount of 20 mg-400 mg per dose, such as, for example, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, 50 mg, 51 mg, 52 mg, 53 mg, 54 mg, 55 mg, 56 mg, 57 mg, 58 mg, 59 mg, 60 mg, 61 mg, 62 mg, 63 mg, 64 mg, 65 mg, 66 mg, 67 mg, 68 mg, 69 mg, 70 mg, 71 mg, 72 mg, 73 mg, 74 mg, 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 96 mg, 97 mg, 98 mg, 99 mg, 100 mg, 101 mg, 102 mg, 103 mg, 104 mg, 105 mg, 106 mg, 107 mg, 108 mg, 109 mg, 110 mg, 111 mg, 112 mg, 113 mg, 114 mg, 115 mg, 116 mg, 117 mg, 118 mg, 119 mg, 120 mg, 121 mg, 122 mg, 123 mg, 124 mg, 125 mg, 126 mg, 127 mg, 128 mg, 129 mg, 130 mg, 131 mg, 132 mg, 133 mg, 134 mg, 135 mg, 136 mg, 137 mg, 138 mg, 139 mg, 140 mg, 141 mg, 142 mg, 143 mg, 144 mg, 145 mg, 146 mg, 147 mg, 148 mg, 149 mg, 150 mg, 151 mg, 152 mg, 153 mg, 154 mg, 155 mg, 156 mg, 157 mg, 158 mg, 159 mg, 160 mg, 161 mg, 162 mg, 163 mg, 164 mg, 165 mg, 166 mg, 167 mg, 168 mg, 169 mg, 170 mg, 171 mg, 172 mg, 173 mg, 174 mg, 175 mg, 176 mg, 177 mg, 178 mg, 179 mg, 180 mg, 181 mg, 182 mg, 183 mg, 184 mg, 185 mg, 186 mg, 187 mg, 188 mg, 189 mg, 190 mg, 191 mg, 192 mg, 193 mg, 194 mg, 195 mg, 196 mg, 197 mg, 198 mg, 199 mg, 200 mg, 201 mg, 202 mg, 203 mg, 204 mg, 205 mg, 206 mg, 207 mg, 208 mg, 209 mg, 210 mg, 211 mg, 212 mg, 213 mg, 214 mg, 215 mg, 216 mg, 217 mg, 218 mg, 219 mg, 220 mg, 221 mg, 222 mg, 223 mg, 224 mg, 225 mg, 226 mg, 227 mg, 228 mg, 229 mg, 230 mg, 231 mg, 232 mg, 233 mg, 234 mg, 235 mg, 236 mg, 237 mg, 238 mg, 239 mg, 240 mg, 241 mg, 242 mg, 243 mg, 244 mg, 245 mg, 246 mg, 247 mg, 248 mg, 249 mg, 250 mg, 251 mg, 252 mg, 253 mg, 254 mg, 255 mg, 256 mg, 257 mg, 258 mg, 259 mg, 260 mg, 261 mg, 262 mg, 263 mg, 264 mg, 265 mg, 266 mg, 267 mg, 268 mg, 269 mg, 270 mg, 271 mg, 272 mg, 273 mg, 274 mg, 275 mg, 276 mg, 277 mg, 278 mg, 279 mg, 280 mg, 281 mg, 282 mg, 283 mg, 284 mg, 285 mg, 286 mg, 287 mg, 288 mg, 289 mg, 290 mg, 291 mg, 292 mg, 293 mg, 294 mg, 295 mg, 296 mg, 297 mg, 298 mg, 299 mg, 300 mg, 301 mg, 302 mg, 303 mg, 304 mg, 305 mg, 306 mg, 307 mg, 308 mg, 309 mg, 310 mg, 311 mg, 312 mg, 313 mg, 314 mg, 315 mg, 316 mg, 317 mg, 318 mg, 319 mg, 320 mg, 321 mg, 322 mg, 323 mg, 324 mg, 325 mg, 326 mg, 327 mg, 328 mg, 329 mg, 330 mg, 331 mg, 332 mg, 333 mg, 334 mg, 335 mg, 336 mg, 337 mg, 338 mg, 339 mg, 340 mg, 341 mg, 342 mg, 343 mg, 344 mg, 345 mg, 346 mg, 347 mg, 348 mg, 349 mg, 350 mg, 351 mg, 352 mg, 353 mg, 354 mg, 355 mg, 356 mg, 357 mg, 358 mg, 359 mg, 360 mg, 361 mg, 362 mg, 363 mg, 364 mg, 365 mg, 366 mg, 367 mg, 368 mg, 369 mg, 370 mg, 371 mg, 372 mg, 373 mg, 374 mg, 375 mg, 376 mg, 377 mg, 378 mg, 379 mg, 380 mg, 381 mg, 382 mg, 383 mg, 384 mg, 385 mg, 386 mg, 387 mg, 388 mg, 389 mg, 390 mg, 391 mg, 392 mg, 393 mg, 394 mg, 395 mg, 396 mg, 397 mg, 398 mg, 399 mg, or 400 mg per dose.

In some aspects, the compound of Formula (I), or pharmaceutically acceptable salt of a compound of Formula (I), is administered intravenously.

In some embodiments, the compound of Formula (I), or pharmaceutically acceptable salt of a compound of Formula (I), is administered intravenously once per week for 3 weeks.

In some embodiments, the compound of Formula (I), or pharmaceutically acceptable salt of a compound of Formula (I), is administered intravenously in cycles, wherein each cycle comprises administering the compound of Formula (I), or pharmaceutically acceptable salt of a compound of Formula (I), once per week for 3 weeks.

In some aspects of the disclosed methods, the compound of Formula (I), or pharmaceutically acceptable salt of a compound of Formula (I), is administered orally.

In some embodiments, the compound of Formula (I), or pharmaceutically acceptable salt of a compound of Formula (I), is administered orally once per week for 3 weeks.

In some embodiments, the compound of Formula (I), or pharmaceutically acceptable salt of a compound of Formula (I), is administered orally in cycles, wherein each cycle comprises administering the compound of Formula (I), or pharmaceutically acceptable salt of a compound of Formula (I), once per week for 3 weeks.

In some aspects of the disclosed methods, the subject is administered a compound of Formula (I).

In other aspects of the disclosed methods, the subject is administered a pharmaceutically acceptable salt of a compound of Formula (I).

In some aspects of the disclosed methods, the subject is administered gemcitabine (or pharmaceutically acceptable salt thereof).

In some embodiments, the subject is administered gemcitabine.

In some embodiments, the subject is administered a pharmaceutically acceptable salt of gemcitabine.

In some embodiments, the subject is administered gemcitabine hydrochloride.

In some aspects of the disclosed methods, the gemcitabine (or pharmaceutically acceptable salt thereof) is administered intravenously.

In some aspects of the disclosed methods, the gemcitabine (or pharmaceutically acceptable salt thereof) is administered in an amount of about 500 mg/m² to about 1500 mg/m² per dose, such as, for example about 500 mg/m², about 505 mg/m², about 510 mg/m², about 515 mg/m², about 520 mg/m², about 525 mg/m², about 530 mg/m², about 535 mg/m², about 540 mg/m², about 545 mg/m², about 550 mg/m², about 555 mg/m², about 560 mg/m², about 565 mg/m², about 570 mg/m², about 575 mg/m², about 580 mg/m², about 585 mg/m², about 590 mg/m², about 595 mg/m², about 600 mg/m², about 605 mg/m², about 610 mg/m², about 615 mg/m², about 620 mg/m², about 625 mg/m², about 630 mg/m², about 635 mg/m², about 640 mg/m², about 645 mg/m², about 650 mg/m², about 655 mg/m², about 660 mg/m², about 665 mg/m², about 670 mg/m², about 675 mg/m², about 680 mg/m², about 685 mg/m², about 690 mg/m², about 695 mg/m², about 700 mg/m², about 705 mg/m², about 710 mg/m², about 715 mg/m², about 720 mg/m², about 725 mg/m², about 730 mg/m², about 735 mg/m², about 740 mg/m², about 745 mg/m², about 750 mg/m², about 755 mg/m², about 760 mg/m², about 765 mg/m², about 770 mg/m², about 775 mg/m², about 780 mg/m², about 785 mg/m², about 790 mg/m², about 795 mg/m², about 800 mg/m², about 805 mg/m², about 810 mg/m², about 815 mg/m², about 820 mg/m², about 825 mg/m², about 830 mg/m², about 835 mg/m², about 840 mg/m², about 845 mg/m², about 850 mg/m², about 855 mg/m², about 860 mg/m², about 865 mg/m², about 870 mg/m², about 875 mg/m², about 880 mg/m², about 885 mg/m², about 890 mg/m², about 895 mg/m², about 900 mg/m², about 905 mg/m², about 910 mg/m², about 915 mg/m², about 920 mg/m², about 925 mg/m², about 930 mg/m², about 935 mg/m², about 940 mg/m², about 945 mg/m², about 950 mg/m², about 955 mg/m², about 960 mg/m², about 965 mg/m², about 970 mg/m², about 975 mg/m², about 980 mg/m², about 985 mg/m², about 990 mg/m², about 995 mg/m², about 1000 mg/m², about 1005 mg/m², about 1010 mg/m², about 1015 mg/m², about 1020 mg/m², about 1025 mg/m², about 1030 mg/m², about 1035 mg/m², about 1040 mg/m², about 1045 mg/m², about 1050 mg/m², about 1055 mg/m², about 1060 mg/m², about 1065 mg/m², about 1070 mg/m², about 1075 mg/m², about 1080 mg/m², about 1085 mg/m², about 1090 mg/m², about 1095 mg/m², about 1100 mg/m², about 1105 mg/m², about 1110 mg/m², about 1115 mg/m², about 1120 mg/m², about 1125 mg/m², about 1130 mg/m², about 1135 mg/m², about 1140 mg/m², about 1145 mg/m², about 1150 mg/m², about 1155 mg/m², about 1160 mg/m², about 1165 mg/m², about 1170 mg/m², about 1175 mg/m², about 1180 mg/m², about 1185 mg/m², about 1190 mg/m², about 1195 mg/m², about 1200 mg/m², about 1205 mg/m², about 1210 mg/m², about 1215 mg/m², about 1220 mg/m², about 1225 mg/m², about 1230 mg/m², about 1235 mg/m², about 1240 mg/m², about 1245 mg/m², about 1250 mg/m², about 1255 mg/m², about 1260 mg/m², about 1265 mg/m², about 1270 mg/m², about 1275 mg/m², about 1280 mg/m², about 1285 mg/m², about 1290 mg/m², about 1295 mg/m², about 1300 mg/m², about 1305 mg/m², about 1310 mg/m², about 1315 mg/m², about 1320 mg/m², about 1325 mg/m², about 1330 mg/m², about 1335 mg/m², about 1340 mg/m², about 1345 mg/m², about 1350 mg/m², about 1355 mg/m², about 1360 mg/m², about 1365 mg/m², about 1370 mg/m², about 1375 mg/m², about 1380 mg/m², about 1385 mg/m², about 1390 mg/m², about 1395 mg/m², about 1400 mg/m², about 1405 mg/m², about 1410 mg/m², about 1415 mg/m², about 1420 mg/m², about 1425 mg/m², about 1430 mg/m², about 1435 mg/m², about 1440 mg/m², about 1445 mg/m², about 1450 mg/m², about 1455 mg/m², about 1460 mg/m², about 1465 mg/m², about 1470 mg/m², about 1475 mg/m², about 1480 mg/m², about 1485 mg/m², about 1490 mg/m², about 1495 mg/m², about or 1500 mg/m² per dose.

In some aspects of the disclosed methods, the gemcitabine (or pharmaceutically acceptable salt thereof) is administered in an amount of 500 mg/m² to 1500 mg/m² per dose, such as, for example 500 mg/m², 505 mg/m², 510 mg/m², 515 mg/m², 520 mg/m², 525 mg/m², 530 mg/m², 535 mg/m², 540 mg/m², 545 mg/m², 550 mg/m², 555 mg/m², 560 mg/m², 565 mg/m², 570 mg/m², 575 mg/m², 580 mg/m², 585 mg/m², 590 mg/m², 595 mg/m², 600 mg/m², 605 mg/m², 610 mg/m², 615 mg/m², 620 mg/m², 625 mg/m², 630 mg/m², 635 mg/m², 640 mg/m², 645 mg/m², 650 mg/m², 655 mg/m², 660 mg/m², 665 mg/m², 670 mg/m², 675 mg/m², 680 mg/m², 685 mg/m², 690 mg/m², 695 mg/m², 700 mg/m², 705 mg/m², 710 mg/m², 715 mg/m², 720 mg/m², 725 mg/m², 730 mg/m², 735 mg/m², 740 mg/m², 745 mg/m², 750 mg/m², 755 mg/m², 760 mg/m², 765 mg/m², 770 mg/m², 775 mg/m², 780 mg/m², 785 mg/m², 790 mg/m², 795 mg/m², 800 mg/m², 805 mg/m², 810 mg/m², 815 mg/m², 820 mg/m², 825 mg/m², 830 mg/m², 835 mg/m², 840 mg/m², 845 mg/m², 850 mg/m², 855 mg/m², 860 mg/m², 865 mg/m², 870 mg/m², 875 mg/m², 880 mg/m², 885 mg/m², 890 mg/m², 895 mg/m², 900 mg/m², 905 mg/m², 910 mg/m², 915 mg/m², 920 mg/m², 925 mg/m², 930 mg/m², 935 mg/m², 940 mg/m², 945 mg/m², 950 mg/m², 955 mg/m², 960 mg/m², 965 mg/m², 970 mg/m², 975 mg/m², 980 mg/m², 985 mg/m², 990 mg/m², 995 mg/m², 1000 mg/m², 1005 mg/m², 1010 mg/m², 1015 mg/m², 1020 mg/m², 1025 mg/m², 1030 mg/m², 1035 mg/m², 1040 mg/m², 1045 mg/m², 1050 mg/m², 1055 mg/m², 1060 mg/m², 1065 mg/m², 1070 mg/m², 1075 mg/m², 1080 mg/m², 1085 mg/m², 1090 mg/m², 1095 mg/m², 1100 mg/m², 1105 mg/m², 1110 mg/m², 1115 mg/m², 1120 mg/m², 1125 mg/m², 1130 mg/m², 1135 mg/m², 1140 mg/m², 1145 mg/m², 1150 mg/m², 1155 mg/m², 1160 mg/m², 1165 mg/m², 1170 mg/m², 1175 mg/m², 1180 mg/m², 1185 mg/m², 1190 mg/m², 1195 mg/m², 1200 mg/m², 1205 mg/m², 1210 mg/m², 1215 mg/m², 1220 mg/m², 1225 mg/m², 1230 mg/m², 1235 mg/m², 1240 mg/m², 1245 mg/m², 1250 mg/m², 1255 mg/m², 1260 mg/m², 1265 mg/m², 1270 mg/m², 1275 mg/m², 1280 mg/m², 1285 mg/m², 1290 mg/m², 1295 mg/m², 1300 mg/m², 1305 mg/m², 1310 mg/m², 1315 mg/m², 1320 mg/m², 1325 mg/m², 1330 mg/m², 1335 mg/m², 1340 mg/m², 1345 mg/m², 1350 mg/m², 1355 mg/m², 1360 mg/m², 1365 mg/m², 1370 mg/m², 1375 mg/m², 1380 mg/m², 1385 mg/m², 1390 mg/m², 1395 mg/m², 1400 mg/m², 1405 mg/m², 1410 mg/m², 1415 mg/m², 1420 mg/m², 1425 mg/m², 1430 mg/m², 1435 mg/m², 1440 mg/m², 1445 mg/m², 1450 mg/m², 1455 mg/m², 1460 mg/m², 1465 mg/m², 1470 mg/m², 1475 mg/m², 1480 mg/m², 1485 mg/m², 1490 mg/m², 1495 mg/m², or 1500 mg/m² per dose.

In some embodiments, the gemcitabine (or pharmaceutically acceptable salt thereof) is administered in an amount of about 1000 mg/m² to about 1250 mg/m² per dose, such as, for example, about 1000 mg/m², about 1005 mg/m², about 1010 mg/m², about 1015 mg/m², about 1020 mg/m², about 1025 mg/m², about 1030 mg/m², about 1035 mg/m², about 1040 mg/m², about 1045 mg/m², about 1050 mg/m², about 1055 mg/m², about 1060 mg/m², about 1065 mg/m², about 1070 mg/m², about 1075 mg/m², about 1080 mg/m², about 1085 mg/m², about 1090 mg/m², about 1095 mg/m², about 1100 mg/m², about 1105 mg/m², about 1110 mg/m², about 1115 mg/m², about 1120 mg/m², about 1125 mg/m², about 1130 mg/m², about 1135 mg/m², about 1140 mg/m², about 1145 mg/m², about 1150 mg/m², about 1155 mg/m², about 1160 mg/m², about 1165 mg/m², about 1170 mg/m², about 1175 mg/m², about 1180 mg/m², about 1185 mg/m², about 1190 mg/m², about 1195 mg/m², about 1200 mg/m², about 1205 mg/m², about 1210 mg/m², about 1215 mg/m², about 1220 mg/m², about 1225 mg/m², about 1230 mg/m², about 1235 mg/m², about 1240 mg/m², about 1245 mg/m², or about 1250 mg/m² per dose.

In some embodiments, the gemcitabine (or pharmaceutically acceptable salt thereof) is administered in an amount of about 1000 mg/m² per dose.

In some embodiments, the gemcitabine (or pharmaceutically acceptable salt thereof) is administered in an amount of about 1250 mg/m² per dose.

In some aspects of the disclosed methods, the gemcitabine (or pharmaceutically acceptable salt thereof) is administered in accordance with the May 2019 Revision (Eli Lilly) of the GEMZAR (gemcitabine) for injection prescribing information.

In some aspects of the disclosed methods, the gemcitabine (or pharmaceutically acceptable salt thereof) is administered in the amounts set forth in the May 2019 Revision (Eli Lilly) of the GEMZAR (gemcitabine) for injection prescribing information.

In some aspects of the disclosed methods, the gemcitabine (or pharmaceutically acceptable salt thereof) is administered according to the dosing times set forth in the May 2019 Revision (Eli Lilly) of the GEMZAR (gemcitabine) for injection prescribing information.

In some aspects of the disclosed methods, the gemcitabine (or pharmaceutically acceptable salt thereof) is administered for the indications set forth in the May 2019 Revision (Eli Lilly) of the GEMZAR (gemcitabine) for injection prescribing information.

In some aspects of the disclosed methods, the gemcitabine (or pharmaceutically acceptable salt thereof) is administered in the combinations set forth in the May 2019 Revision (Eli Lilly) of the GEMZAR (gemcitabine) for injection prescribing information.

In some aspects of the disclosed methods, the gemcitabine (or pharmaceutically acceptable salt thereof) is administered by intravenous infusion over a period of about 10 minutes to about 60 minutes, such as, for example, about 10 min, about 15 min, about 20 minr, about 25 min, about 30 min, about 35 min, about 40 min, about 45 min, about 50 min, about 55 min, or about 60 min.

In some embodiments, the gemcitabine (or pharmaceutically acceptable salt thereof) is administered by intravenous infusion over about 30 minutes.

In some embodiments, the gemcitabine (or pharmaceutically acceptable salt thereof) is administered by intravenous infusion over 30 minutes.

In some aspects of the disclosed methods, the gemcitabine (or pharmaceutically acceptable salt thereof) is administered by intravenous infusion on two days per 21-day cycle.

In other aspects of the disclosed methods, the gemcitabine (or pharmaceutically acceptable salt thereof) is administered by intravenous infusion on three days per 28-day cycle.

In other aspects of the disclosed methods, the gemcitabine (or pharmaceutically acceptable salt thereof) is administered by intravenous infusion once weekly.

In other aspects of the disclosed methods, the gemcitabine (or pharmaceutically acceptable salt thereof) is administered by intravenous infusion once weekly for 7 weeks.

In other aspects of the disclosed methods, the gemcitabine (or pharmaceutically acceptable salt thereof) is administered by intravenous infusion once weekly for 3 weeks of each 28-day cycle.

In other aspects of the disclosed methods, the gemcitabine (or pharmaceutically acceptable salt thereof) is administered by intravenous infusion once weekly for the first 7 weeks, then one week rest, then once weekly for 3 weeks of each 28-day cycle.

In other aspects of the disclosed methods, the gemcitabine (or pharmaceutically acceptable salt thereof) is administered by intravenous infusion over 30 minutes on Days 1 and 8 of each 21-day cycle.

In other aspects of the disclosed methods, the gemcitabine (or pharmaceutically acceptable salt thereof) is administered by intravenous infusion over 30 minutes on Days 1 and 8 of each 21-day cycle.

In other aspects of the disclosed methods, the gemcitabine (or pharmaceutically acceptable salt thereof) is administered by intravenous infusion over 30 minutes on Days 1, 8, and 15 of each 28-day cycle

In other aspects of the disclosed methods, the gemcitabine (or pharmaceutically acceptable salt thereof) is administered by intravenous infusion over 30 minutes on Days 1 and 8 of each 21-day cycle.

In other aspects of the disclosed methods, the gemcitabine (or pharmaceutically acceptable salt thereof) is administered by intravenous infusion over 30 minutes once weekly for the first 7 weeks, then one week rest, then once weekly for 3 weeks of each 28-day cycle.

In some aspects, the treatment regimen of the disclosure further comprise an additional therapeutic agent. Exemplary additional therapeutic agents include, for example, other anticancer agents, such as, for example, cisplatin, carboplatin, or paclitaxel.

In some aspects, the treatment regimen of the disclosure further comprises cisplatin.

In some aspects, the treatment regimen of the disclosure further comprises carboplatin.

In some aspects, the treatment regimen of the disclosure further comprises paclitaxel.

In some aspects, the methods of the disclosure are directed to treating cancer in a subject. In some embodiments, the cancer is one or more of include squamous-cell carcinoma, basal cell carcinoma, adenocarcinoma, hepatocellular carcinomas, and renal cell carcinomas, cancer of the bladder, bowel, breast, cervix, colon, esophagus, head, kidney, liver, lung, neck, ovary, pancreas, prostate, and stomach; leukemias; benign and malignant lymphomas, particularly Burkitt's lymphoma and Non-Hodgkin's lymphoma; benign and malignant melanomas; myeloproliferative diseases; sarcomas, including Ewing's sarcoma, hemangiosarcoma, Kaposi's sarcoma, liposarcoma, myosarcomas, peripheral neuroepithelioma, synovial sarcoma, gliomas, astrocytomas, oligodendrogliomas, ependymomas, gliobastomas, neuroblastomas, ganglioneuromas, gangliogliomas, medulloblastomas, pineal cell tumors, meningiomas, meningeal sarcomas, neurofibromas, and Schwannomas; bowel cancer, breast cancer, prostate cancer, cervical cancer, uterine cancer, lung cancer, ovarian cancer, testicular cancer, thyroid cancer, astrocytoma, esophageal cancer, pancreatic cancer, stomach cancer, liver cancer, colon cancer, melanoma; carcinosarcoma, Hodgkin's disease, Wilms' tumor and teratocarcinomas. Additional cancers which may be treated using compounds according to the present disclosure include, for example, T-lineage Acute lymphoblastic Leukemia (T-ALL), T-lineage lymphoblastic Lymphoma (T-LL), Peripheral T-cell lymphoma, Adult T-cell Leukemia, Pre-B ALL, Pre-B Lymphomas, Large B-cell Lymphoma, Burkitts Lymphoma, B-cell ALL, Philadelphia chromosome positive ALL and Philadelphia chromosome positive CML.

In other embodiments, the cancer is one or more of breast cancer, non-small cell lung cancer, lung squamous cell carcinoma, uterine/endometrio adenocarcinoma, ovarian cancer, lung adenocarcinoma, bladder cancer, skin cancer, hormone refractory prostate cancer, colon adenocarcinoma, gastric adenocarcinoma, pancreatic cancer, AIDS-related Kaposi's sarcoma, or squamous cell carcinoma of the head and neck cancer.

In other embodiments, the cancer is one or more of breast cancer, non-small cell lung cancer, esophageal cancer, ovarian cancer, or pancreatic cancer.

In some embodiments, the cancer is breast cancer or prostate cancer.

In some embodiments, the cancer is breast cancer.

In some embodiments, the cancer is metastatic breast cancer.

In some embodiments, the cancer is prostate cancer.

In some embodiments, the cancer is hormone-refractory metastatic prostate cancer.

In some embodiments, the cancer is non-small cell lung cancer.

In some embodiments, the cancer is adenocarcinoma of the pancreas.

In other embodiments, the cancer is breast cancer.

In other embodiments, the cancer is non-small cell lung cancer.

In other embodiments, the cancer is ovarian cancer.

In other embodiments, the cancer is pancreatic cancer.

In some embodiments of the disclosed methods, the cancer is a SMARCA4 deficient cancer. As used herein, the term “SMARCA4 deficient” means that the cancer either lacks a SMARCA4 gene, or has a mutated SMARCA4 gene that encodes a fully- or partially-non-functional SMARCA4 protein.

In some embodiments of the disclosed methods, the cancer is not a SMARCA4 deficient cancer.

In some aspects of the methods of the disclosure, administration of the disclosed treatment regimen results in greater tumor growth inhibition than results from administration of either Formula (I) alone or gencitabine alone. As used herein, tumor growth inhibition is calculated as follows: Mean % Inhibition=(mean(C)−mean(T))/mean(C)*100%, wherein C is the tumor volume in the control arm, and T is the tumor volume in the treatment arm.

In some aspects of the methods of the disclosure, administration of the disclosed treatment regimen results in at least 40% tumor growth inhibition, such as, for example, at least 40%, at least 45%, at least 50%, at least 55%, at least 60%, at least 65%, at least 70%, at least 75%, at least 80%, at least 85%, at least 90%, or at least 95% tumor growth inhibition, relative to no treatment.

In some aspects of the methods of the disclosure, the cancer exhibits a complete response (CR) or a partial response (PR) to the administration of the treatment regimen, as evaluated by the Response Evaluation Criteria in Solid Tumors (RECIST) v1.1 criteria.

In some embodiments, the cancer exhibits a complete response (CR) to the administration of the treatment regimen, as evaluated by the Response Evaluation Criteria in Solid Tumors (RECIST) v1.1 criteria.

In other embodiments, the cancer exhibits a partial response (PR) to the administration of the treatment regimen, as evaluated by the Response Evaluation Criteria in Solid Tumors (RECIST) v1.1 criteria.

The disclosure is also directed to the following aspects:

Aspect 1. A method of treating cancer in a subject in need thereof, comprising administering to the subject a therapeutically effective amount of a SMARCA2 inhibitor.

Aspect 2. The method of aspect 1, wherein the SMARCA2 inhibitor comprises Compound I, or a pharmaceutically acceptable salt thereof.

Aspect 3. The method of any one of the preceding aspects, wherein the SMARCA2 inhibitor is administered using a dosing regimen and schedule disclosed herein.

Aspect 4. The method of any one of the preceding aspects, wherein the cancer comprises one or more tumors with loss of SMARCA4.

Aspect 5. The method of any one of the preceding aspects, wherein the cancer comprises one or more tumors with mutated SMARCA4.

### EXAMPLES

### Example 1

A study is conducted to evaluate the anti-tumor efficacy of the Formula (I)+gemcitabine combination in SMARCA4 deficient human lung cancer xenogtaft models in mice. Specific objectives include:
1. To determine whether the Formula (I)+gemcitabine combination in SMARCA4 deficient lung cancer models in mice results in deeper tumor growth inhibition (TGI) compared to each single agent alone.
2. To determine whether the Formula (I)+gemcitabine combination in SMARCA4 deficient lung cancer models in mice results in deeper tumor growth inhibition (TGI) compared to each single agent alone.

### Materials

### Formula (I)

Formula (I) was stored in powder form at −20° C. The compound was dissolved in 100% DMSO to make a 10 mM stock. The dissolved DMSO solution was stored at −20° C.

Gemcitabine (Jiangsu Hansoh Pharmaceutical Group Co., Ltd. Lot #: H20030104)

### Experimental Procedures

### NCI-H838 Tumor Bearing Mouse Model

The NCI-H838-#5 tumor cells are maintained in vitro with RPMI1640 medium supplemented with 10% fetal bovine serum at 37° C. in an atmosphere of 5% CO₂ in air. The cells in exponential growth phase are harvested and quantitated by cell counter before tumor inoculation. NOD-SCID female mice are inoculated subcutaneously in the right front flank region with NCI-H838-#5 tumor cells (5×10⁶) in 0.1 ml of PBS mixed with Matrigel (1:1) for tumor development. The randomization is performed when the mean tumor size reaches approximately 100-150 mm³. Tumor volumes are measured every other day after randomization in two dimensions using a caliper, and the volume are expressed in mm³ using the formula: “V=(L×W×W)/2, where V is tumor volume, L is tumor length (the longest tumor dimension) and W is tumor width (the longest tumor dimension perpendicular to L).

### Data Analysis

Tumor volumes at the end of studies are evaluated by the Mann-Whitney test (unpaired, nonparametric test). *P<0.05, **P<0.01 versus vehicle are shown in graph.

### Results

Formula (I) combination with gemcitabine results in deeper tumor growth inhibition (TGI) compared to each single agent alone in NCI-H838 SMARCA4 deficient tumors at well tolerated doses.

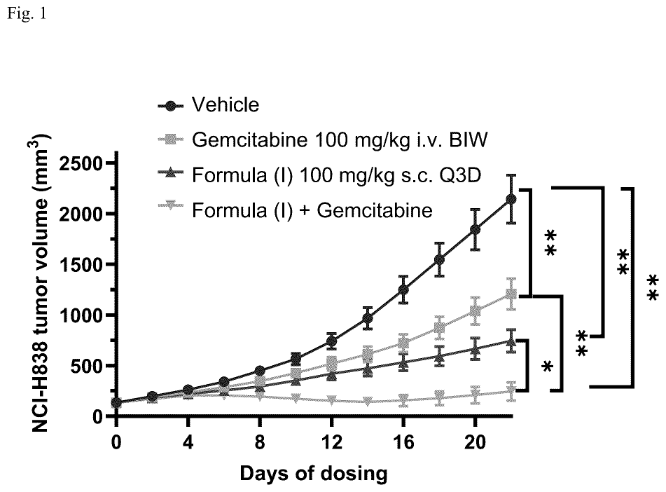

The efficacy of Formula (I) and gemcitabine on tumor growth inhibition is evaluated in SMARCA4 deficient lung cancer models in mice. For the NCI-H838 CDX model, mice are inoculated subcutaneously with the NCI-H838 tumor cells (5×10⁶). Formula (I) administered subcutaneously (s.c.) every third day (Q3D) at 100 mg/kg significantly inhibits the growth of NCI-H838 tumors. Bi-weekly (BIW) intravenous (i.v.) administration of gemcitabine at 100 mg/kg also shows significant tumor growth inhibition in this model. The combination of Formula (I) (s.c., Q3D, 100 mg/kg) and gemcitabine (i.v., BIW, 100 mg/kg) results in a significantly higher TGI compared to each single agent alone. See FIG. 1.

1. A method of treating cancer in a subject in need thereof, comprising administering to the subject a treatment regimen comprising:
(a) a compound of Formula (I): 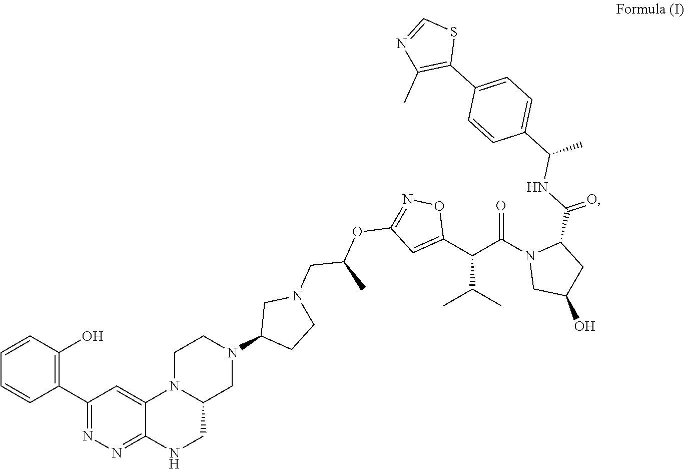 [SMILES: Cc1ncsc1-c1ccc([C@H](C)NC(=O)[C@@H]2C[C@@H](O)CN2C(=O)[C@@H](c2cc(O[C@@H](C)CN3CC[C@@H](N4CCN5c6cc(-c7ccccc7O)nnc6NC[C@H]5C4)C3)no2)C(C)C)cc1]
or a pharmaceutically acceptable salt thereof; and
(b) gemcitabine, or a pharmaceutically acceptable salt thereof.

2. The method of claim 1, wherein the compound of Formula (I), or a pharmaceutically acceptable salt thereof, and the gemcitabine, or a pharmaceutically acceptable salt thereof, are administered concurrently or sequentially.

3. The method of claim 1, wherein the subject is administered the compound of Formula (I), or a pharmaceutically acceptable salt of a compound of Formula (I) (on a Formula (I) basis), in an amount of about 20 mg-about 1000 mg per dose.

4. The method of claim 1, wherein the compound of Formula (I), or pharmaceutically acceptable salt of a compound of Formula (I), is administered intravenously.

5. The method of claim 1, wherein the compound of Formula (I), or pharmaceutically acceptable salt of a compound of Formula (I), is administered once per week for 3 weeks.

6. The method of claim 1, wherein the subject is administered a compound of Formula (I).

7. The method of claim 1, wherein the subject is administered a pharmaceutically acceptable salt of a compound of Formula (I).

8. The method of claim 1, wherein the gemcitabine, or a pharmaceutically acceptable salt thereof, is administered intravenously.

9. The method of claim 1, wherein the gemcitabine (or pharmaceutically acceptable salt thereof) is administered in an amount of about 500 mg/m² to about 1500 mg/m² per dose.

10. The method of claim 8, wherein the gemcitabine (or pharmaceutically acceptable salt thereof) is administered by intravenous infusion over a period of about 10 minutes to about 60 minutes.

11. The method of claim 8, wherein the gemcitabine (or pharmaceutically acceptable salt thereof) is administered by intravenous infusion on two days per 21-day cycle.

12. The method of claim 8, wherein the gemcitabine (or pharmaceutically acceptable salt thereof) is administered by intravenous infusion on three days per 28-day cycle.

13. The method of claim 8, wherein the gemcitabine (or pharmaceutically acceptable salt thereof) is administered by intravenous infusion once weekly.

14. The method of claim 8, wherein the gemcitabine (or pharmaceutically acceptable salt thereof) is administered by intravenous infusion once weekly for 7 weeks.

15. The method of claim 8, wherein the gemcitabine (or pharmaceutically acceptable salt thereof) is administered by intravenous infusion once weekly for 3 weeks of each 28-day cycle.

16. The method of claim 8, wherein the gemcitabine (or pharmaceutically acceptable salt thereof) is administered by intravenous infusion once weekly for the first 7 weeks, then one week rest, then once weekly for 3 weeks of each 28-day cycle.

17. The method of claim 8, wherein the gemcitabine (or pharmaceutically acceptable salt thereof) is administered by intravenous infusion over 30 minutes on Days 1 and 8 of each 21-day cycle.

18. The method of claim 8, wherein the gemcitabine (or pharmaceutically acceptable salt thereof) is administered by intravenous infusion over 30 minutes on Days 1 and 8 of each 21-day cycle.

19. The method of claim 8, wherein the gemcitabine (or pharmaceutically acceptable salt thereof) is administered by intravenous infusion over 30 minutes on Days 1, 8, and 15 of each 28-day cycle

20. The method of claim 8, wherein the gemcitabine (or pharmaceutically acceptable salt thereof) is administered by intravenous infusion over 30 minutes on Days 1 and 8 of each 21-day cycle.

21. The method of claim 8, wherein the gemcitabine (or pharmaceutically acceptable salt thereof) is administered by intravenous infusion over 30 minutes once weekly for the first 7 weeks, then one week rest, then once weekly for 3 weeks of each 28-day cycle.

22. The method of claim 1, wherein the treatment regimen further comprises an additional therapeutic agent.

23. The method of claim 22, wherein the additional therapeutic agent is cisplatin, carboplatin, or paclitaxel.

24. The method of claim 1, wherein the cancer is one or more of breast cancer, non-small cell lung cancer, esophageal cancer, ovarian cancer, or pancreatic cancer.

25. The method of claim 1, wherein the cancer is a SMARCA4 deficient cancer.

26. The method of claim 1, wherein administration of the treatment regimen results in greater tumor growth inhibition than results from administration of either Formula (I) alone or the taxane anticancer agent alone.

27. The method of claim 1, wherein administration of the treatment regimen results in at least 40% tumor growth inhibition relative to no treatment.

28. The method of claim 1, wherein the cancer exhibits a complete response (CR) or a partial response (PR) to the administration of the treatment regimen, as evaluated by the Response Evaluation Criteria in Solid Tumors (RECIST) v1.1 criteria.

29. The method of claim 28, wherein the cancer exhibits a complete response (CR) to the administration of the treatment regimen, as evaluated by the Response Evaluation Criteria in Solid Tumors (RECIST) v1.1 criteria.

30. The method of claim 28, wherein the cancer exhibits a partial response (PR) to the administration of the treatment regimen, as evaluated by the Response Evaluation Criteria in Solid Tumors (RECIST) v1.1 criteria.
